(19) United States
(12) Patent Application Publication
(10) Pub. No.: US 2010/0174328 A1
(43) Pub. Date: Jul. 8, 2010

(54) Method and compostion for repair and reconstruction of intervertebral discs and other reconstructive surgery

(76) Inventors: James P. Seaton, JR., Chatham, NJ (US); Linda Trebing, Madison, NJ (US)

Correspondence Address: Daniel Pollmann, 1507 W. Parkside Lane, Phoenix, AZ, 85027, US

(21) Appl. No.: 12/655,788

(22) Filed: Jan. 7, 2010

Related U.S. Application Data
(62) Division of application No. 11/602,810, filed on Nov. 21, 2006, now abandoned.
(60) Provisional application No. 60/738,857, filed on Nov. 22, 2005.

(51) Int. Cl. A61B 17/56; A61F 2/44

(52) U.S. Cl. 606/86 R; 623/17.16

(57) ABSTRACT

By providing an elastic form stable material which is capable of being delivered directly to a specific desired location within a living creature and providing increased strength and rigidity to the injected location, disorders of the intervertebral disc of a living creature are able to be effectively treated. Treatment of defects or voids in soft tissue is achieved with a variation of the subject material specific to each application. In the preferred method, the elastic form stable material is injected directly into the affected area, thereby achieving the desired result.

### CROSS-REFERENCE TO RELATED PATENT APPLICATIONS

Pursuant to 35 U.S.C. Section 120, this application is related to and claims benefit of U.S. Provisional Patent Application Ser. No. 60/738,857, filed Nov. 22, 2005, entitled METHOD AND COMPOSITION FOR REPAIR AND RECONSTRUCTION OF INTERVERTEBRAL DISCS AND OTHER RECONSTRUCTIVE SURGERY. Pursuant to 35 U.S.C. Section 121, this application claims benefit of the earlier filing date and is a DIVISIONAL Patent Application of U.S. Non-Provisional patent application Ser. No. 11/602,810, filed Nov. 21, 2006, entitled METHOD AND COMPOSITION FOR REPAIR AND RECONSTRUCTION OF INTERVERTEBRAL DISCS AND OTHER RECONSTRUCTIVE SURGERY.

### BACKGROUND OF THE INVENTION

The present invention relates to a method of injecting a polymer to treat disordered, insufficient, or injured structures in a living creature, in particular, a human being. This invention also relates to a composition suitable for use in such a method, its preparation and use.

Many structures in the human body rely upon the flexibility or elasticity of the tissue to exhibit the preferred properties or to perform a desired task. The resilience of the tissue often provides a means for better load distribution and shock-absorbing characteristics. The elasticity may function to create a more natural appearance or more desirable tactile property. Specific examples of these types of structures include the intervertebral discs, or the connective and soft tissue beneath the skin, often associated with providing shape and resilience for the external, visible parts of the body (i.e., maxillofacial).

Intervertebral Disc

The spinal column is composed of 24 vertebrae, which are stacked on top of each other. The individual bony vertebral bodies are separated by spinal discs; these soft structures serve several functions: maintaining the proper spacing and alignment between the vertebrae, absorbing and distributing loads on the vertebrae. The intervertebral disc has a strong fibrous outer ring called the annulus and a softer, gelatinous center called the nucleus pulposus. The annulus surrounds and contains the nucleus and serves as a strong ligament that connects the adjacent vertebrae. The nucleus pulposus provides a shock absorbing mechanism for the spine. The disc also aids in maintaining the appropriate disc height to prevent nerve root impingement by adjacent bony spinal structures.

As the body ages, the disc loses much of its natural cushioning properties through the loss of fluids and elasticity. The annulus may develop small cracks and tears, which can be painful. Annular tears may weaken the fibrous structure to the point that the nucleus pulposus is no longer sufficiently contained and bulges out, or herniates, into the spinal canal or other structures. This pressure on the spinal cord or nerve roots from the herniation can cause severe pain and can eventually lead to impairment of bodily function or paralysis. A herniated disc can also be caused by placing forces on the spine during standard daily activities—moving, lifting, sneezing, or by traumatic events.

The disc height and resilience can be significantly reduced by the loss or dehydration of nucleus material. The loss of the “shock-absorbing” ability of the intervertebral discs can increase the peak loads seen by the adjacent vertebral bodies. In addition, the aging population often exhibits osteoporosis which can weaken the vertebral body, increasing the risk for potential vertebral compression fractures.

Current treatments for the aging intervertebral disc, annular tears, and disc herniation include removal of herniated material through mechanical, laser, or chemical means, insertion of pre-formed nucleus replacement devices, repair of the annulus by suturing or changing the structure of the fibers. The disc may be completely removed and replaced with an articulating prosthesis or a stationary spinal spacer to fuse the adjacent vertebrae. All of the options have the same primary goal, which is pain relief at the affected level, allowing the patient to regain mobility. However, these treatments affect the natural mechanics of the disc by changing the properties of the annulus or nucleus, or by completely replacing the structure(s).

What is proposed is a therapy which would allow the clinician to inject a flowable substance which will polymerize in situ to mimic many of the desired characteristics of the affected structure. The polymer could be tailored to produce a tougher material to fill or repair tears or weaknesses in the annulus, and a softer, more resilient material would be applied to augment or replace the nucleus pulposus.

Plastic Surgery

Plastic surgery applications include the filling of voids in soft tissue or bone that may have occurred due to tumor or cyst removal, trauma, or deformity correction. The application of the material would provide underlying support and structure for the affected areas, and the characteristics of the implant could be tailored for functional or cosmetic purposes. The durometer of the polymer can be chosen to better match or augment the tissue that is being filled, replaced or reconstructed.

It may also be desirable to select a compliant or flexible material for void filling or defect repair in bony structures which are adjacent to soft tissue structures that may be subject to swelling or edema. The flexibility of the implant could allow a pressure release or reduction mechanism during the healing and swelling period. An example would be as a bur hole cover in the skull. In addition, the compliant, rubber-like nature of this implant will also allow more accessibility to the underlying tissue for biopsy, aspiration of fluids, etc.

### SUMMARY OF THE INVENTION

By employing the present invention, all of the difficulties and drawbacks found in the prior art have been eliminated and a highly effective method for treating diseased, injured, or disordered structures, particularly intervertebral discs, in living creatures is attained. In addition, the present invention also achieves a unique composition particularly formulated and suitable for use in the method of the present invention.

The method of the present invention comprises the injection of an elastomeric filler into the structure to be treated via a percutaneous route, usually under X-ray guidance, such as lateral projection fluoroscopy. The material is injected as a paste or semi-liquid from a suitable gun or injection system via a needle that has been passed into body to apply the material to the affected area. The elastomeric filler, once injected, will polymerize in situ. The resulting material provides reinforcement to or replacement of tissue or anatomical structures that are deficient due to the aging process, tumor removal or other surgical intervention, trauma. In addition to the reinforcing, strengthening and shock-absorbing properties, it is desirable that the starting filler composition is of a viscosity that allows it to flow into the voids or spaces as required.

Therefore, it is a principal object of the present invention to provide filler material and a method for using the filler material into an intervertebral disc, the substrate beneath the cartilage in articulating joints, the voids in bony or cartilaginous structures created or treated during plastic or neurosurgical procedures, or other similar applications, which is easily prepared and delivered to the affected area while also providing the desired filling, reinforcing, strengthening and shock-absorbing properties.

Another object of the present invention is to provide filler material and a method for using the filler material in the affected areas having the characteristic features described above which is inherently flexible and viscous to provide flowability throughout the structures as required both during its application and after curing, thereby achieving self-regulating control realized from the fluid properties of the injected liquid and the elastomeric characteristics of the polymerized material.

Another object of the present invention is to provide filler material and a method for using the filler material in the body having the characteristic features described above which is capable of being prepared to exhibit varying levels of hardness or stiffness after curing, thereby allowing the selection and formulation of the filler material with appropriate mechanical properties specifically suited for each application.

Another object of the present invention is to provide filler material and a method for using the filler material in the body having the characteristic features described above which is capable of being prepared with additives that remain active for a length of time after curing, thereby allowing localized therapeutic treatment of the affected area or anatomic structure.

Another object of the present invention is to provide a method for application of the filler material in the body in a controlled and directed manner that results in the placement of the material in targeted areas and having a specific geometry that is preferred for the treatment modality.

Other and more specific objects will in part be obvious and will in part appear hereinafter.

### THE DRAWINGS

For a fuller understanding of the nature and objects of the invention, reference should be had to the following detailed description taken in connection with the accompanying drawings, in which:

FIG. 1 is a front perspective view, partially broken away, depicting the anatomy of a spinal column with one associated intervertebral disc; and

FIGS. 2-4 are rear perspective views, partially broken away, depicting various stages in the method of the present invention.

### DETAILED DESCRIPTION OF THE INVENTION

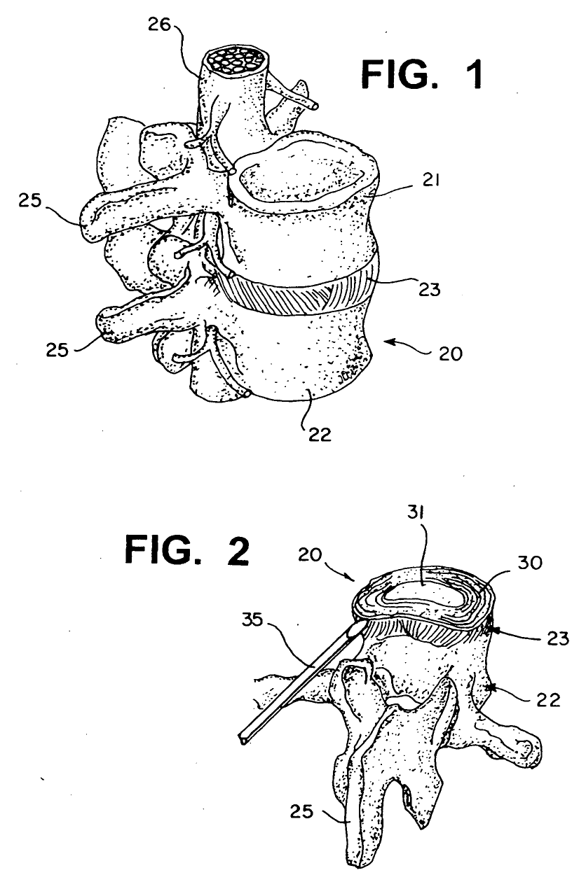

By referring to FIGS. 1-4, along with the following detailed disclosure, one of the principal treatment methods of the present invention can best be understood. In this regard, as detailed above, the present invention can be implemented in many areas of a human body. However, one principal application of the present invention is in the repair of intervertebral discs. In addition, the unique formulation of the material employed in the method of the present invention is also fully disclosed. However, alterations or variations in both the method and the formulation of the material can be made without departing from the scope of the present invention. Consequently, it is to be understood that the following detailed discussion and the drawings are provided for exemplary purposes only and are not intended as a limitation of the present invention.

In FIG. 1, a portion of a conventional spinal column 20 is depicted, with vertebral bodies 21 and 22 shown incorporating intervertebral disc 23 interposed therebetween. Each vertebral body 21 and 22 incorporates bony structure 25 extending therefrom along with spinal cord 26 axially extending along the entire length of spinal column 20, in nested protective engagement with bony structures 25.

As detailed above, each intervertebral disc 23 maintains the proper spacing and alignment between vertebral bodies 21 and 22, while also absorbing and distributing loads imposed upon the vertebrae. Due to aging, injury, and excessive loads, intervertebral disc 23 often incurs a wide variety of injuries or physical degradation loses, causing the disc to lose much of its natural cushioning properties. In addition, cracks or tears in the disc structure weaken the fibrous structure forming the disc, often causing the disc to deform, bulge, or herniate into the spinal canal or other structures. These various maladies cause severe pain, as well as leading to an impairment of various bodily functions.

By employing the present invention, any damaged or impaired intervertebral disc 23 is capable of being repaired in a direct, easily implemented process. As detailed herein, a needle or cannula is inserted into the damaged or impaired vertebral disc 23 and a uniquely formulated, curable, filler composition is injected into the interior of the intervertebral disc. In the preferred embodiment, the curable filler material comprises an elastic form stable material which is allowed to cure or polymerize in situ, effectively reforming the damaged intervertebral disc 23 and curing the damages or impairments originally present in disc 23.

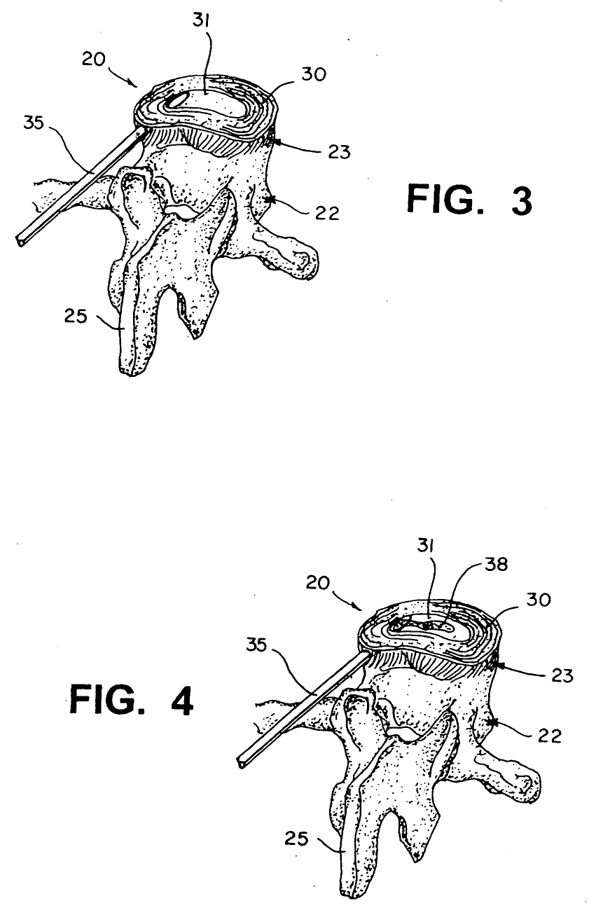

By referring to FIGS. 2-4, along with the following detailed discussion, the method of the present invention can best be understood. As shown therein, intervertebral disc 23 comprises a strong fibrous outer ring or annulus 30 and a softer, gelatinous center or nucleus pulposus 31. Annulus 30 surrounds and contains nucleus pulposus 31 and serves as a strong ligament that connects the adjacent vertebrae. In addition, nucleus pulposus 31 provides a shock absorbing mechanism for the spine, with disc 23 maintaining the appropriate height to prevent nerve root impingement by adjacent bony spinal structures 25.

As discussed above, due to the aging process and/or injury, nucleus pulposus 31 often loses fluids and elasticity, thereby losing much of its natural cushioning properties. Furthermore, annulus 30 often develops cracks or tears, which weaken the fibrous structure thereof to a sufficient extent that nucleus pulposus 31 is no longer sufficiently contained within annulus 30.

These problems frequently occur, resulting in various physical difficulties, including severe pain, impairment of bodily functions, impairment of daily activities and/or paralysis. However, by employing the present invention, intervertebral disc 23 can be repaired and the physical difficulties eliminated or substantially reduced.

In accordance with the present invention, a flowable, curable filler composition comprising an elastic form stable material is injected directly into nucleus pulposus 31 and allowed to cure in situ. Once the elastic form stable material has polymerized, the material mimics the physical characteristics inherently present in nucleus pulposus 31.

In this way, all of the physical difficulties or impairments suffered by the individual are virtually eliminated or substantially reduced. In addition, by forming the flowable, curable filler composition of the present invention to impart a tougher material to disc 23 when cured, tears or weaknesses in annulus 30 of disc 23 are able to be repaired.

As shown in FIG. 2, in employing the method of the present invention, needle or cannula 35 is inserted into the individual to be treated and positioned directly adjacent annulus 30 of disc 23 to be repaired. Once in the proper position, needle/cannula 35 is advanced through annulus 30 with the tip thereof entering nucleus pulposus 31, as depicted in FIG. 3. Thereafter, as shown in FIG. 4, curable filler composition 38 is advanced through needle/cannula 35 into nucleus pulposus 31, filling or augmenting the area defined by nucleus pulposus 31 in order to provide the improved and enhanced properties thereto. Once the desired quantity of filler material 38 has been added into nucleus pulposus 31, needle/cannula 35 is withdrawn and filler material 38 is allowed to cure in order to provide the desired enhanced beneficial results to disc 23.

In accordance with the present invention, it has been found that the preferred curable filler composition comprises an elastic form stable material. Preferably, this material comprises a silicone elastomer, with poly (dimethyl siloxane) being preferred. In addition, in the preferred formulation, the composition also incorporates a cross-linking agent and a diluent.

One composition of the curable filler material of the present invention which has been found to be extremely successful comprises between about 60% and 85% by weight based upon the weight of the entire composition of poly (dimethyl siloxane), between about 2% and 5% by weight based upon the weight of the entire composition of a cross-linking agent, and between about 10% and 20% by weight based upon the weight of the entire composition of a diluent. In addition, if desired, a radiopaque material may be incorporated into the composition in order to enable the delivery of the material into disc 23 to be monitored by using suitable equipment, such as x-rays.

In this regard, it has been found that between about 10% and 20% by weight based upon the weight of the entire composition of the radiopaque material is preferably employed. In addition, the radiopaque material preferably comprises one selected from the group consisting of silver powder, barium sulfate, bismuth trioxide, zirconium dioxide, tantalum or titanium powders or fibers, calcium sulfate, calcium phosphate, hydroxyapatite, tri-calcium phosphate, and other medically appropriate opacifier agents.

One preferred formulation of the “cure-in-place” silicone elastomer of the present invention comprises two highly viscous liquid components, namely reinforced dimethyl methylvinyl siloxanes and reinforced dimethyl methylhydrogen siloxanes, supplied in equal parts (1:1 ratio). In addition, this preferred composition is preferably supplied in a pre-filled, two-part mixing and dispensing cartridge/syringe system wherein the two viscous liquid components are maintained separately until the time of use. When desired, the components are thoroughly intermixed with each other in the desired uniform ratio to achieve the desired uniform consistency.

Once the mixing process has been completed, the resulting silicone elastomer is immediately injected into the desired site wherein the material polymerizes in situ in approximately 3 to 15 minutes. Once cured, the silicone elastomer results in a tough, rubbery consistency which has low toxicity and presents a low risk of unfavorable biological reactions. In addition, the preferred formulation preferably incorporates a radio-opaque material in order to enable the delivery of the material to be monitored with standard fluoroscopy.

In accordance with the present invention, it has been found at the following compositions represents the preferred formulations for the two component system of this invention:

Component A:
Vinyldimethyl terminated dimethyl polysiloxane/trimethylsiloxy terminated polydimethyl siloxane, 64%
Silica, amorphous, 21%
Barium Sulfate powder, USP, 15%
Pt Catalyst>0.001%

Component B:
Vinyldimethyl terminated dimethyl polysiloxane/trimethylsiloxy terminated polydimethyl siloxane, 63%
Silica, amorphous, 21%
Barium Sulfate powder, USP, 15%
Trimethyl methyl-hydro dimethyl siloxane (crosslinker), 1%

By employing the compositions detailed above, all of the desired goals and objectives of the present invention are realized.

It has also been found that the curable filler material employed in the method of the present invention preferably possesses a durometer in the cured state which ranges between about 10 A and 90 A. In addition, it has also been found that the curable filler material can be delivered to disc 23 in two stages, with the first stage being employed to fill cracks or tears in annulus 30, while the second stage is employed to repair nucleus pulposus 31. In this regard, the second stage material is delivered to disc 23 after the first stage material has cured. By employing this process, assurance is provided that the material supplied during the second stage is completely retained in disc 23, since any damage or maladies in annulus 23 are fully repaired prior to the delivery of the material for the second stage.

If desired, it has been found that the delivery of the curable filler material to disc 23 in two stages can be achieved in a manner which closely resembles or mimics the normal, anatomical construction. In this regard, the first stage of the repair of annulus 30 is achieved by employing filler material resulting in a durometer in the cured state which ranges between about 30 A and 90 A. In addition, in the second stage, which is employed to repair nucleus pulposus 31, the filler material employed comprises a durometer in the cured state which ranges between about 10 A and 50 A. By employing this dual durometer or differential material construction, the natural or normal anatomical configuration is realized, with a softer, more flexible, and more compressible material forming nucleus pulposus 31 while being peripherally surrounded by a stronger material forming annulus 30.

It has also been found that the present invention can be implemented by employing a hydrogel as the material for forming nucleus pulposus 31. In this regard, the silicone elastomer detailed above is employed for forming the first stage or repairing annulus 30 in the manner detailed above. Once cured, the second stage of the repair is implemented by injecting a hydrogel into annulus 30 forming nucleus pulposus 31. In this way, the desired disc repair is achieved in a manner which achieves all of the desired goals and objectives.

It has also been found that the method of the present invention can be employed in combination with a disc oblation procedure. In this regard, a void is created within intervertebral disc 23 and, once established, the curable filling material of the present invention is supplied thereto. Furthermore, it has also been found that the process of the present invention can be employed in combination with a balloon kyphoplasty procedure or similar deployment of an expandable device or with a steerable biopsy needle or instrument. In this procedure, an expandable balloon or similar device is inserted into disc 23 as a replacement or reinforcement for annulus 30. Once in position, the curable filler material of the present invention is delivered into the balloon or expandable device for providing the filler material thereto, or to inject the material and expand a balloon to compress the material outward toward the annulus forming a reinforced surface. Alternatively, a steerable instrument, such as an articulating or flexible needle or catheter may be used to coat the interior surface of the annulus thus reinforcing it.

Although the foregoing detailed disclosure has focused on the use of the present invention in connection with the repair or reconstruction of a disordered intervertebral disc, the present invention has equal applicability and efficacy in other surgical areas, as discussed above. In this regard, plastic surgery represents another specific area where the method and material composition and formulations of the present invention is equally applicable. Consequently, all of the foregoing detailed disclosure is hereby repeated and reiterated herein, with complete applicability to these other areas where the same overall procedures and material formulations can be employed.

It will thus be seen that the object set forth above, among those made apparent from the preceding description, are efficiently attained and, since certain changes may be made in carrying out the above method and in the composition set forth without departing from the scope of the invention, it is intended that all matter contained in the above description or shown in the accompanying drawings shall be interpreted as illustrative and not in a limiting sense.

It is also to be understood that the following claims are intend to cover all of the generic and specific features of the invention herein described, and all statements of the scope of the invention which, as a matter of language, might be said to fall therebetween.

Particularly, it is to be understood that in said claims, ingredients or compounds recited in the singular are intended to include compatible mixtures of such ingredients wherever the sense permits.

Having described our invention, what we claim is:

1. A method for treating a disease or injured intervertebral disc in a human being comprising the steps of:
A. preparing a first curable filler material in advance of its use, said first filler material being formulated to possess a durometer when cured which ranges between about 30 A and 90 A;
B. preparing a second curable filler material and advance of its use, said second filler material being formulated to possess a durometer when cured which ranges between about 10 A and 50 A;
C. inserting a first needle or cannula into the damaged or impaired intervertebral disc for enabling said first filler material to be delivered to said disc;
D. causing said first filler material to flow through said needle or cannula into the damaged or impaired intervertebral disc for enabling said first filler material to fill cracks or tears therein;
E. withdrawing the needle or cannula from the damaged or impaired intervertebral disc;
F. allowing said first filler material to cure to the desired durometer level;
G. inserting a second needle or cannula into the damaged or impaired intervertebral disc for enabling said second filler material to be delivered to said disc;
H. causing said second filler material to flow through said needle or cannula into the damaged or impaired intervertebral disc for enabling said second filler material to fill the interior of said disc and provide the desired soft and compressible material for achieving the desired repair of said disc.

2. The method defined in claim 1, wherein said first curable filler material and said second curable filler material are further defined as being prepared by intermixing:
a. between about 60% and 85% by the way based upon the weight of the entire composition of poly (dimethyl siloxane),
b. between about 2% and 5% by weight based upon the weight of the entire composition of a cross-linking agent,
c. between about 10% and 20% by weight based upon the weight of the entire composition of diluent, and
d. between about 10% and 20% by weight based upon the weight of the entire composition of a radiopaque material.

3. The method defined in claim 1, wherein the first curable filler material and the second curable filler material adhere to each other after curing, forming an inseparable fond therebetween.

4. The method defined in claim 1, in which the first filler material is directed to fill or repair cracks or voids in an annulus of the intervertebral disc or to reinforce or strengthen a weakened or herniated area of the annulus.

5. The method defined in claim 4, in which the durometer of resulting cured material is selected to provide increased strength and stability to the disc annulus.

6. The method defined in claim 5, wherein the second filler material fills a central portion of the intervertebral disc, typically occupied by the nucleus pulposus in a healthy, non-diseased intervertebral disc.

7. The method defined in claim 6, in which the durometer of the second filler material is selected to provide increased flexibility, resilience, or a greater capacity to distribute forces seen by the intervertebral disc.

8. The method defined in claim 7, in which the durometer of cured second filler material is different from the durometer of the first cured filler material.

9. The method defined in claim 6, wherein a desired amount of first filler material is removed to provide a void within the annulus.

10. The method defined in claim 4, wherein the second material filler is injected into the void created.

11. The method defined in claim 1, in which the injection of the curable filler material is applied in combination with a fibrous or mesh structure, such that the fibrous or mesh structure serves to contain an injectable filler material within the intervertebral disc.

12. The method defined in claim 1, in which the injection of the curable filler material is applied in combination with a disc ablation procedure, such that a void is created within the intervertebral disc for the curable filler material.

13. The method defined in claim 1, in which the injection of the curable filler material is applied in combination with a balloon kyphoplasty procedure, or similar deployment of an expandable device, such that a void is created within the intervertebral disc for the curable filler material.

14. The method defined in claim 1, in which the injection of the curable filler material is applied in combination with application of sutures or surgical closure devices, such that the surgical closure devices seal the periphery of the intervertebral disc and serve to contain the injectible filler material within the intervertebral disc.

15. The method defined in claim 1, in which the injection of the curable filler material is applied in combination with application of fibrin glue devices, such that the fibrin glue devices seal the periphery of the intervertebral disc and serve to contain the injectable filler material within the intervertebral disc.

16. The method defined in claim 1, wherein the filler material is directionally delivered by using an articulating or steerable needle or catheter.

17. The method defined in claim 6, wherein a hydrogel material is injected during a second stage into the central portion of a repaired intervertebral disc.
